(19) United States
(12) Patent Application Publication
(10) Pub. No.: US 2014/0378329 A1
(43) Pub. Date: Dec. 25, 2014

(54) Animal Colony Monitoring Using Fecal Samples

(71) Applicant: IDEXX Laboratories, Inc., Westbrook, ME (US)

(72) Inventor: Matthew Howard Myles, Moberly, MO (US)

(21) Appl. No.: 14/311,663

(22) Filed: Jun. 23, 2014

Related U.S. Application Data
(60) Provisional application No. 61/838,530, filed on Jun. 24, 2013.
(60) Provisional application No. 61/863,751, filed on Aug. 8, 2013.

(51) Int. Cl. G01N 33/58

(52) U.S. Cl. CPC G01N 33/582; G01N 2469/20
USPC 506/9; 506/13

(57) ABSTRACT

A library of fecal samples from animals populations for analysis of disease in the population and methods for collection and analysis of the samples. Methods for managing a population of animals using the library and methods.

### CROSS-REFERENCE TO RELATED APPLICATIONS

This application claims the benefit of U.S. Provisional Patent Application Ser. No. 61/863,751, filed Aug. 8, 2013, and U.S. Provisional Patent Application Ser. No. 61/838,530, filed Jun. 24, 2013, each of which are incorporated by reference herein in its entirety.

### FIELD

The disclosure relates to the collection and analysis of biological samples from populations of animals. More particularly, the disclosure is directed to the health management of animal colonies using fecal samples from the members of the colony for analysis a phenotype of the colony or of exposure of the colony to infectious agents.

### BACKGROUND

Over the past five decades, great strides have been made in the identification and eradication of infections from laboratory rodents. As a result, most contemporary biomedical research rodent colonies are relatively free of the pathogenic viruses, parasites, bacteria, and fungi that cause clinical disease. However, some microbes, especially those agents that cause subclinical disease, remain in an enzootic state in many research colonies. These agents, despite their insidious nature, have an impact on physiologic parameters of the host and thus on the results of animal experiments, independent of their pathogenic potential. Therefore, timely and accurate diagnosis of infectious disease in animal research models is critical to the success of biomedical research. To this end, institutional veterinarians closely monitor the health of research animals through periodic systematic examination of sample groups of research animals against a predetermined list of infectious agents. Rodent health monitoring can generally be accomplished using a combination of molecular and serological diagnostic assays. Molecular diagnostic tools provide a real-time assessment of infection; whereas, serological tools detect the presence of antibodies to infectious agents, thus, providing an historical perspective of infectious disease exposure over the life of the animal.

The current practice for collection of blood or serum for serological evaluation of infectious disease in laboratory animals includes either euthanasia of animals for collection of blood by cardiocentesis or ante mortem blood collection from the mandibular, lateral saphenous or retro orbital vein. Once collected the whole blood sample is allowed to clot, which typically requires 2-12 hours, then whole blood is centrifuged and the serum is separated from the cellular (clotted) fraction. Next, the serum is shipped to a facility at refrigerated or frozen temperatures using an overnight service (generally one or two pounds of ice packs are required) in a STRYOFOAM™ shipping box.

This practice is inconvenient and expensive in light of the amount of animal colonies and the number of analytes that must be tested to ensure colony health and homogeneity. Accordingly, the inventors have identified a need in the art to provide a simplified and efficient method for rodent colony health monitoring and management.

### SUMMARY

In one aspect, the disclosure is directed to a library of fecal samples from a population of animals. The library includes a plurality of sample vessels containing fecal samples from the population of animals. Each sample vessel may identify the animal or colony of animals providing the sample, and the vessels may be bundled to be transported as a single unit to a laboratory for determining information regarding one or more biological markers in the population of animals. In various embodiments of the disclosure, the population of animals may be a rodent colony. Also the biological markers may be antibodies or infectious agents that induce antibodies present in the fecal samples.

In another aspect, the disclosure is directed to a method of analyzing a biological marker in a population of animals. The method includes collecting a fecal sample from two or more members of a population of animals; transporting the fecal samples to a laboratory as a single unit; processing the samples to extract mucosal antibody from the fecal samples; and analyzing the processed samples for the presence or absence of at least one antibody. In various embodiments of the disclosure, the samples may be collected following seroconversion of an animal in the population, or for example, at 1, 2, 3, 4, 5, or 6 weeks following infection of an animal in the population. The analysis of the samples may include a multiplex immunoassay for antibodies extracted from the feces. The assay may be designed and configured to detect at least ten different antibodies. In another embodiment, the samples are analyzed for the presence at least one infectious agent antibody to the agent.

In a further aspect, the disclosure is directed to a method of health monitoring and managing a rodent colony. The method includes analyzing biological samples from each member or groups of members of the rodent colony and verifying the presence or absence of the at least one biological marker in the colony. The method may also include removing members from the colony that test positive or negative for the biological marker.

In yet another aspect, the disclosure is directed a method of determining a presence or absence of an infectious disease in a population of rodents. The method includes providing a plurality of fecal collection containers to a user responsible for a population of rodents. The method also includes providing instructions to the user comprising the following: (i) collect a representative number of fecal samples from individual rodents in a population or cage samples from cages containing a plurality of members of the population; (ii) place the individual fecal samples or the cage samples in individual fecal collection containers for each sample; (iii) transport the plurality of collection containers to a laboratory as a single unit. The method further includes receiving the plurality of containers as a single unit from the user, processing the samples to extract mucosal antibody from the samples, and analyzing the extracted samples for a presence or absence an antibody to an infectious agent, thereby determining the presence or absence of an infectious disease in the population. The results of the presence or absence of the infectious disease in the population are reported to the user.

### DESCRIPTION

The disclosure addresses the challenges associated with the collection, identification and processing of voluminous numbers of samples obtained from animal colonies that are well known in the life sciences. The colonies usually include small animals, such as rodents, felines, canines, and rabbits, but the disclosure is applicable, for example, to animal colonies where large numbers of samples are required to be tested in order to monitor the health of the colonies. These colonies are maintained for research purposes and, in many cases, it is critical that the members of the colonies have particular phenotypes and health status. The testing of individual members of the colonies for up 50 different bacterial and viral agents and for desired phenotypic characteristics may be necessary to ensure the health of the colony and its usefulness in testing, for example, the efficacy of pharmaceutical agents on large populations.

The method of the disclosure provides that, for colony sampling purposes, only fecal samples need to be collect from colony animals and/or cages; thus, altogether negating or reducing the need for blood draws and significantly simplifying the process of rodent colony health monitoring. This disclosure makes live (antemortem) sample collection safer, simpler and more feasible (eliminates the need for phlebotomists therefore enabling expanded or self-sampling). Accordingly, in one aspect, the disclosure provides a library of fecal samples from a population of animals. The library includes samples from a plurality of members of a colony. Typically, a statically significant number of animals in the colony are tested for particular phenotype or disease. For example, in very large populations (e.g., up to 100,000 animals) as many to 400 animals are tested (e.g., 2 animals per group of 50). In some populations, all of the animals are tested. Collection of the test samples from the individual members of the colony provides a library of samples representative of the population.

In accordance with the disclosure, the library of fecal samples is collected directly from individual animals or from the cage and placed in collection containers or vessels, for example 1.5 ml conical micro-centrifuge tubes (typically 2-4 fecal pellets per tube). Once the samples from the colony have been collected, they can be transported as a single unit containing a plurality of sample vessels representing the colony or a subset thereof, using commercially available transportation and delivery services (e.g., U.S. Mail, FEDEX®, UPS®) in standard delivery envelops without refrigeration to a reference laboratory for analysis. Many analytes are stable in fecal samples for several or more days. Of particular interest, antibodies are generally stable for up to 14 days at room temperature.

At the clinical laboratory, the fecal samples are treated with an appropriate buffer to extract the desired markers. In the case of small molecules, the solvent can be anything that is a solvent for the analyte. Methanol and acetonitrile are widely used, either straight or mixed with water. Water itself may also be used for extremely polar analytes. Extraction of ionizable analytes is often improved by pH adjustment—increasing the charge to improve solubility in water or reducing it to promote solubility in organic solvents. In some instances, extraction of even moderately polar analytes is increased by adding about 10-15% water to methanol, sometimes by adding water to the sample first, then allowing to soak a few minutes before adding organic solvent. An extremely hydrophobic analyte may be best extracted with a nonpolar solvent such as hexane, also providing some cleanup by leaving polar contaminants undissolved in the punch.

For antibodies, peptides and proteins, aqueous buffers with pH and salt concentration to promote protein stability can be used. The addition of a non-ionic detergent, such as 0.1% TWEEN™-20 or TRITON™ X-100 detergents may be desirable. Removal may require incubation with gentle mixing for one to several hours. Hydrophobic peptides may extract better with some methanol or acetonitrile added.

In one embodiment, fecal antibodies are eluted with 250 microliters of buffer containing Tris-buffered saline with 1 mM EDTA. The sample container may be placed in the refrigerator overnight to allow efficient antibody elution. Once eluted, the fecal slurry may be centrifuged at 25,000×g for 20 minutes to remove particulate material.

Once the library samples are extracted, the samples are analyzed for the presence or absence of a biological marker. For example, mouse colonies can be tested for the following infectious agents or antibodies to infectious agents, and the samples may be tested in various subsets (panels) as exemplified in Table 1.

TABLE 1

| | Mouse Panel | | | | | |
|---|---|---|---|---|---|---|
| Test | A | B | C | D | E | F |
| MHV | X | X | X | X | X | X |
| MVM (MMV) | X | X | X | X | X | X |
| NS1 (Generic Parvovirus) | X | X | X | X | X | X |
| MPV (MPV1-5) | X | X | X | X | X | X |
| MNV | X | X | X | X | X | X |
| TMEV | X | X | X | X | X | X |
| EDIM | X | X | X | X | X | X |
| Sendai virus | | X | X | X | X | X |
| Mycoplasma pulmonis | | X | X | X | X | X |
| PVM | | | X | X | X | X |
| REO3 | | | X | X | X | X |
| LCMV | | | X | X | X | X |
| Ectromelia virus | | | X | X | X | X |
| MAD1 | | | | X | X | X |
| MAD2 | | | | X | X | X |
| Polyoma virus | | | | X | X | X |
| Encephalitozoon cuniculi | | | | | X | X |
| CARB | | | | | X | X |
| Clostridium piliforme | | | | | X | X |
| MCMV | | | | | X | X |
| K virus | | | | | | X |
| Hantaan virus | | | | | | X |
| Lactate dehydrogenase-elevating virus | | | | | | X |
| MTV(IFA) | | | | | | X |

Similarly, for rat colonies, the analytes and panels are exemplified in Table 2.

TABLE 2

| | Rodent Panel | | | | |
|---|---|---|---|---|---|
| Test | A | B | C | D | E |
| RCV | X | X | X | X | X |
| NS1 (Generic Parvovirus) | X | X | X | X | X |
| RPV | X | X | X | X | X |
| RMV | X | X | X | X | X |
| KRV | X | X | X | X | X |
| H-1 | X | X | X | X | X |
| RTV (Rat theilovirus) | X | X | X | X | X |
| Sendai virus | | X | X | X | X |
| PVM | | X | X | X | X |
| Mycoplasma pulmonis | | X | X | X | X |
| REO3 | | | X | X | X |
| LCMV | | | X | X | X |
| CARB | | | | X | X |
| Hantaan virus | | | | X | X |
| Clostridium piliforme | | | | X | X |
| MAD1 | | | | X | X |
| MAD2 | | | | | X |
| Encephalitozoon cuniculi | | | | | X |
| IDIR | | | | | X |

Animal colonies of other species can be analyzed for panels of markers appropriate for the species. Typically, the fecal samples are analyzed for antibodies that are induced by the markers (e.g., infectious agent), which indicates that the animal is infected with the the agent. In some embodiments, the markers, including antibodies, can be detected directly. Therefore, as used herein, the marker can either be an infectious agent, e.g., antigen, or an antibody to agent/antigen.

Overall sensitivity is very much a function of the analyte, matrix interferences, chromatography conditions and the evaluation methods. In general, antibodies and proteins can be extracted from fecal samples to provide a concentration range of about 0.1-10 ng/mL.

In general, very few viral agents are persistently shed in feces, and PCR is must be employed for detecting most viral agents during a short (usually 1-2 week) shedding period. Antibodies are persistent long term, and allow the use of fecal samples for the detection of infection throughout the life of the animal. Antibodies, however, tend to be prevalent in feces only after a seroconversion of the animal. Animals are usually seropositive one to two weeks, and up to six weeks, after infection. Accordingly, in one aspect the disclosure is directed to the detection of antibodies in feces following seroconversion of one or more animals in a population, which is typically 1, 2, 3, 4, 5 or 6 weeks following an infection. Samples can be collected from the population at various intervals to ensure that infected animals are removed from the population as soon as disease is identified. In another aspect, feces may be tested by PCR for antigen in conjunction with immunoassays for antibodies in order to identify recent or historic infection of one or more animals in the population.

In one aspect, the extracted samples are analyzed in a Multiplex Fluorescent Immunoassay (MFI) that is based both on bead-based immunoassay and flow cytometry. Purified antigen or control preparations are covalently linked to one of, for example, 100 different types of polystyrene beads, which vary slightly in the intensity of their color. If IgG antibody to a particular antigen is present, then it will bind to the antigen on a specific bead and will then be detected by subsequent binding of goat anti-species antibody conjugated to a fluorochrome (e.g., R-phycoerythrin). The reader channels single beads through a dual laser detector which simultaneously determines both the bead type by the internal dye combination and the fluorescent intensity associated with each individual bead. The fluorescent intensity associated with each of the individual beads of each type is used in the determination of each MFI value. Side-by-side testing of thousands of individual results from hundreds of samples show overall correlation between MFI and ELISA is greater than 99.5% for both mouse and rat samples. In general, MFI is more sensitive than ELISA and is less prone to false positive results. MFI requires only about 1.0 μL to 10 μL of sample regardless of the number of tests requested.

The ability to use small sample sizes for testing several analytes in a sample using MFI sample coupled with this disclosure including library of fecal samples allows for the comprehensive and convenient analysis of a colony of animals. The results of the colony analysis can be transmitted directed to the colony manager by electronic communications, including, cloud, e-mail and smart phone applications, so that that laboratory manager has immediate access to data regarding the colony or individual room of a colony.

In other embodiments, the eluent is then evaluated by other known immunoassay techniques known to those of skill in the art (e.g., IFA and western blot).

In one aspect, the disclosure is directed to a method of managing a rodent colony. The method analysis of biological markers for the exposure to infectious agents, disease or phenotype within the colony using fecal samples, and sample collection and analysis as described herein. Colony management may include removing members from the colony that test positive or negative for the biological marker.

In another embodiment, the disclosure is directed to method of determining the presence or absence of an infectious disease in a population of rodents. The method includes providing a plurality of fecal collection containers to a user responsible for a population of rodents. A user responsible for a population of rodents is, for example, the population manager, technician, veterinarian, or other person who cares for, control, or manages the care and wellbeing of the population, The method also includes providing instructions to the user container the following instructions:
(i) collect a representative number of fecal samples from individual rodents in a population or cage samples from cages containing a plurality of members of the population;
(ii) place the individual fecal samples or the cage samples in individual fecal collection containers for each sample;
(iii) transport the plurality of collection containers to a laboratory as a single unit;

After the user receives the containers and instructions, and carries out the instructions, the plurality of containers is received as a single unit from the user. The samples are processed to extract mucosal antibody from the samples; and processed samples are analyzed for a presence or absence an antibody to an infectious agent. Once the presence or absence of an infectious disease in the population is identified as a result of the presence or absence of antibodies in the samples, the results may be reported to the user.

The following are provided for exemplification purposes only and are not intended to limit the scope of the disclosure described in broad terms above. All references cited in this disclosure are incorporated herein by reference.

### EXAMPLES

### Example 1

### Monitoring a Rodent Colony for Infectious Agents

For example, routine rodent health monitoring for infectious agent exposure is accomplished by evaluating rodent fecal samples for the presence of antibodies formed as part of the immune response to infection. To accomplish this, fecal samples are collected from individual animals or from cages housing a number of animals and placed in 1.5 ml conical micro-centrifuge tubes. Once the fecal samples from the rodent research colony have been collected, they are transported, using commercially available transportation and delivery services (e.g., U.S. Mail, FEDEX® or UPS®) in standard delivery envelops without refrigeration, to a reference laboratory for analysis. For shipping efficiency, samples from more than one animal and/or more than one colony can be gathered shipped together as a single unit. Samples are tested against a predetermined list of markers of an infection (see, for example, Table 1 and Table 2, above) and results are reported to the submitter. When an infectious disease outbreak is detected, the infected animals are identified using the unique identification codes and quarantined. Additional steps may be taken to ascertain the extent of the outbreak and to eliminate/control the infectious agent. Ultimately, it may be necessary to re-derive or restock the colony with disease-free animals.

### Example 2

Feces were collected in micro-centrifuge tubes from a colony of rodents housed in six cages. Representative samples were collected individually from 24 rodents and cage samples were obtained for each of the six cages. Samples were placed in microcentrifuge tubes and the tubes were labeled with the appropriate animal and/or cage identifier. The tubes were shipped as a single unit containing a library of samples to a reference laboratory where samples were extracted and tested for antibodies to MHV and MPV using Multiplex Fluorescent Immunoassay (MFI) as described above. Results are shown in Table 3.

TABLE 3

| ID | Client ID | Cage | Sample Type | MHV | MPV |
|---|---|---|---|---|---|
| 1 | Right Upper Punch | Cage 1 | Mouse feces | + | − |
| 2 | Right Lower Punch | Cage 1 | Mouse feces | + | − |
| 3 | Left upper Punch | Cage 1 | Mouse feces | + | − |
| 4 | Left Lower Punch | Cage 1 | Mouse feces | + | − |
| 5 | Right Upper Punch | Cage 2 | Mouse feces | + | − |
| 6 | Right Lower Punch | Cage 2 | Mouse feces | + | − |
| 7 | Left Lower Punch | Cage 2 | Mouse feces | − | − |
| 8 | Left Lower Punch | Cage 2 | Mouse feces | + | − |
| 9 | Right Upper Punch | Cage 3 | Mouse feces | + | − |
| 10 | Right Lower Punch | Cage 3 | Mouse feces | + | − |
| 11 | Left Upper Punch | Cage 3 | Mouse feces | + | − |
| 12 | Left Lower Punch | Cage 3 | Mouse feces | + | − |
| 13 | | Cage 1 | Cage feces | + | − |
| 14 | | Cage 1 | Cage feces | + | − |
| 15 | | Cage 2 | Cage feces | + | − |
| 16 | | Cage 2 | Cage feces | + | − |
| 17 | | Cage 3 | Cage feces | + | − |
| 18 | | Cage 3 | Cage feces | − | + |
| 19 | Right Upper Punch | Cage 4 | Mouse feces | + | − |
| 20 | Right Lower Punch | Cage 4 | Mouse feces | − | + |
| 21 | Left Upper Punch | Cage 4 | Mouse feces | + | + |
| 22 | Left Lower Punch | Cage 4 | Mouse feces | + | − |
| 23 | Right Upper Punch | Cage 5 | Mouse feces | − | − |
| 24 | Right Lower Punch | Cage 5 | Mouse feces | + | − |
| 25 | Left upper Punch | Cage 5 | Mouse feces | + | − |
| 26 | Left Lower Punch | Cage 5 | Mouse feces | − | + |
| 27 | Right Upper Punch | Cage 6 | Mouse feces | − | + |
| 28 | Right Lower Punch | Cage 6 | Mouse feces | − | + |
| 29 | Left Upper Punch | Cage 6 | Mouse feces | − | + |
| 30 | Left Lower Punch | Cage 6 | Mouse feces | − | + |
| 31 | | Cage 4 | Cage feces | − | + |
| 32 | | Cage 4 | Cage feces | − | + |
| 33 | | Cage 5 | Cage feces | − | − |
| 34 | | Cage 5 | Cage feces | − | + |
| 35 | | Cage 6 | Cage feces | − | + |
| 36 | | Cage 6 | Cage feces | − | − |

Results were provided to the colony manager and used to determine how to manage the colony in light of the results. Management included removal from the colony the animals that tested positive for the marker(s).

The disclosures of all references and publications cited herein are expressly incorporated by reference in their entireties to the same extent as if each were incorporated by reference individually.

Although various specific embodiments of the invention have been described herein, it is to be understood that the invention is not limited to those precise embodiments and that various changes or modifications can be affected therein by one skilled in the art without departing from the scope and spirit of the invention.

The examples given above are merely illustrative and are not meant to be an exhaustive list of all possible embodiments, applications or modifications of the invention. Thus, various modifications and variations of the described methods and systems of the invention will be apparent to those skilled in the art without departing from the scope and spirit of the invention. Although the invention has been described in connection with specific embodiments, it should be understood that the invention as claimed should not be unduly limited to such specific embodiments.

What is claimed is:

1. A library of fecal samples from a population of animals, comprising a plurality of sample vessels containing fecal samples from the population of animals, wherein each sample vessel identifies the animal or colony of animals providing the sample, and wherein the vessels are bundled to be transported as a single unit to a laboratory for determining information regarding one or more biological markers in the population of animals.

2. The library of claim 1, where the population of animals is a rodent colony.

3. The library of claim 1, wherein the biological markers are antibodies.

4. The library of claim 1, wherein the biological markers are infectious agents that induce antibodies present in the fecal samples.

5. A method of analyzing a biological marker in an population of animals, the method comprising:
(a) collecting a fecal sample from two or more members of a population of animals;
(b) transporting the fecal samples to a laboratory as a single unit;
(c) processing the samples to extract mucosal antibody from the fecal samples; and
(d) analyzing the processed samples for the presence or absence of at least one antibody.

6. The method of claim 5, wherein the samples are collected following seroconversion of an animal in the population.

7. The method of claim 5, wherein the samples are collected at 1, 2, 3, 4, 5, or 6 weeks following infection of an animal in the population.

8. The method of claim 5, wherein the analyzing comprises conducting a multiplex immunoassay for antibodies extracted from the feces.

9. The method of claim 8, wherein the multiplex immunoassay is designed and configured to detect at least ten different antibodies.

10. The method of claim 5, wherein samples are analyzed for the presence at least one infectious agent and antibody to the agent.

11. A method of health monitoring and managing a rodent colony:
(a) analyzing biological samples from each member or groups of members of the rodent colony according to the method of claim 5, and
(b) verifying the presence or absence of the at least one biological marker in the colony.

12. The method of claim 11, further comprising removing members from the colony that test positive or negative for the biological marker.

13. A method of determining a presence or absence of an infectious disease in a population of rodents, the method comprising:
(a) providing a plurality of fecal collection containers to a user responsible for a population of rodents;
(b) providing instructions to the user comprising the following:
(i) collect a representative number of fecal samples from individual rodents in a population or cage samples from cages containing a plurality of members of the population;
(ii) place the individual fecal samples or the cage samples in individual fecal collection containers for each sample;
(iii) transport the plurality of collection containers to a laboratory as a single unit;
(c) receiving the plurality of containers as a single unit from the user,
(c) processing the samples to extract mucosal antibody from the samples;
(d) analyzing the extracted samples for a presence or absence an antibody to an infectious agent, thereby determining the presence or absence of an infectious disease in the population; and
(f) reporting the results of the presence or absence of the infectious disease in the population to the user.

14. The method of claim 13, wherein the samples are collected following seroconversion of an animal in the population.

15. The method of claim 13, wherein the samples are collected at 1, 2, 3, 4, 5, or 6 weeks following infection of an animal in the population.

16. The method of claim 13, wherein the analyzing comprises conducting a multiplex immunoassay for antibodies extracted from the feces.

17. The method of claim 16, wherein the multiplex immunoassay is designed and configured to detect at least ten different antibodies.

18. The method of claim 13, wherein the instructions further comprise, prior to instruction (iii), the following instruction: identify at least one of the rodent population and the individual rodent on each of the plurality of collection containers.
